(19) United States
(12) Patent Application Publication
(10) Pub. No.: US 2010/0076198 A1
(43) Pub. Date: Mar. 25, 2010

(54) Crystalline forms of Fentanyl Alkaloid

(75) Inventor: Gary A. Nichols, Wildwood, MO (US)

Correspondence Address: Mallinckrodt Inc., 675 McDonnell Boulevard, HAZELWOOD, MO, 63042, US

(73) Assignee: Mallinckrodt Inc., Hazelwood, MO (US)

(21) Appl. No.: 12/560,614

(22) Filed: Sep. 16, 2009

Related U.S. Application Data
(60) Provisional application No. 61/098,264, filed on Sep. 19, 2008.

(51) Int. Cl. C07D 211/32

(52) U.S. Cl. 546/234

(57) ABSTRACT

The present invention provides crystalline forms of fentanyl alkaloid and methods for preparing crystalline forms of fentanyl alkaloid.

### FIELD OF THE INVENTION

The present invention generally relates to crystalline forms of fentanyl alkaloid and processes for preparing crystalline forms of fentanyl alkaloid.

### BACKGROUND OF THE INVENTION

Solids exist in either amorphous or crystalline forms. In the case of crystalline forms, molecules are positioned in three-dimensional lattice sites. When a compound recrystallizes from a solution or slurry, it may crystallize with different spatial lattice arrangements, and the different crystalline forms are sometimes referred to as “polymorphs.” The different crystalline forms of a given substance may differ from each other with respect to one or more chemical properties (e.g., dissolution rate, solubility), biological properties (e.g., bioavailability, pharmacokinetics), and/or physical properties (e.g., mechanical strength, compaction behavior, flow properties, particle size, shape, melting point, degree of hydration or salvation, caking tendency, compatibility with excipients). The variation in properties among different crystalline forms usually means that one crystalline form is desired or preferred over other forms.

Fentanyl, N-[1-(2-phenylethyl)-4-piperidyl]-N-phenylpropanamide, is an opioid analgesic that was first synthesized in the early 1960s (Janssen, 1062, Br. J. Anaesth. 34:260-268). Fentanyl is characterized by a very high potency (approximately about eighty times that of morphine), a rapid onset, and a short duration of action. Fentanyl is used extensively as an analgesic or anesthetic, most often in operating rooms and intensive care units, and the fentanyl transdermal system is used in chronic pain management. Fentanyl transdermal systems or patches frequently comprise fentanyl alkaloid embedded in a gel or a matrix for sustained release. Despite the widespread use of fentanyl alkaloid and the possibility that different crystalline forms of fentanyl alkaloid may provide beneficial chemical or biological properties, no crystalline forms of fentanyl alkaloid, however, have been characterized. A need exists, therefore, for new crystalline forms of fentanyl alkaloid, as well as processes for the preparation of the different crystalline forms of fentanyl alkaloid.

### SUMMARY OF THE INVENTION

The present invention provides crystalline forms of fentanyl alkaloid and processes for producing the different crystalline forms of fentanyl alkaloid. Among the various aspects of the invention is a provision for a crystalline form of fentanyl alkaloid, N-[1-(2-phenylethyl)-4-piperidyl]-N-phenylpropanamide, the crystalline form being Form II.

Another aspect of the invention encompasses a pharmaceutical composition comprising crystalline Form II of fentanyl alkaloid, N-[1-(2-phenylethyl)-4-piperidyl]-N-phenylpropanamide, and at least one pharmaceutically acceptable excipient.

A further aspect of the invention provides a process for preparing a substantially pure crystalline form of fentanyl alkaloid, N-[1-(2-phenylethyl)-4-piperidyl]-N-phenylpropanamide. The process comprises contacting fentanyl alkaloid with a solvent to form a saturated or near saturated solution, and evaporating the solvent in the solution to form a mass of crystals of the substantially pure crystalline form of fentanyl alkaloid.

Still another aspect of the invention encompasses a process for converting a crystalline Form I of fentanyl alkaloid into a crystalline Form II of fentanyl alkaloid. The process comprises melting the crystalline Form I of fentanyl alkaloid, cooling the melted fentanyl alkaloid, and heating the cooled fentanyl alkaloid to form the crystalline Form II of fentanyl alkaloid.

Other aspects and features of the invention will be in part apparent and in part described in more detail below.

### DESCRIPTION OF THE FIGURES

FIG. 1 presents an X-ray powder diffraction pattern of crystalline Form I of fentanyl alkaloid. Peak intensity is plotted as a function of degrees 2-theta.

FIG. 2 presents a differential scanning calorimetry thermogram of crystalline Form I of fentanyl alkaloid. Heat flow is plotted as a function of temperature.

FIG. 3 presents an X-ray powder diffraction pattern of crystalline Form II of fentanyl alkaloid. Peak intensity is plotted as a function of degrees 2-theta.

FIG. 4 presents a differential scanning calorimetry thermogram of crystalline Form II of fentanyl alkaloid. Heat flow is plotted as a function of temperature.

FIG. 5 presents an X-ray powder diffraction pattern of crystalline Form III of fentanyl alkaloid. Peak intensity is plotted as a function of degrees 2-theta

### DETAILED DESCRIPTION

It has been discovered that fentanyl alkaloid, whose chemical name is N-[1-(2-phenylethyl)-4-piperidyl]-N-phenylpropanamide, may exist as any of several crystalline forms that differ from each other with respect to their physical properties, spectral data, stability, and methods of preparation. Three crystalline forms of fentanyl alkaloid are described herein, and are hereinafter referred to, respectively, as Form I, Form II, and Form III. Form I is the predominate crystalline form in fentanyl alkaloid produced by Mallinckrodt Inc. (St. Louis, Mo.). Form II is a new crystalline form that is not observed in the above-mentioned production material. Form III is a meta-stable form that is observed only at extremely low temperatures. The present invention also provides a pharmaceutical composition comprising crystalline Form II of fentanyl alkaloid and at least one pharmaceutically acceptable excipient. Also provided are processes for producing crystalline Forms I and II, as well as a process for the conversion of Form I into Form II.

### (I) Crystalline Forms of Fentanyl Alkaloid

A first aspect of the invention encompasses three crystalline forms of fentanyl alkaloid. The three crystalline forms may be distinguished on the basis of different X-ray powder diffraction patterns. The two crystalline forms (i.e., Form I and Form II) that are observed at room temperature also may be distinguished on the basis of different endothermic transitions or melting temperatures, as determined by differential scanning calorimetry. Those of skill in the art will appreciate that other analytical techniques, such as single crystal X-ray diffraction analysis, Fourier transform infrared spectroscopy, etc., also may be used to distinguish these crystalline forms.

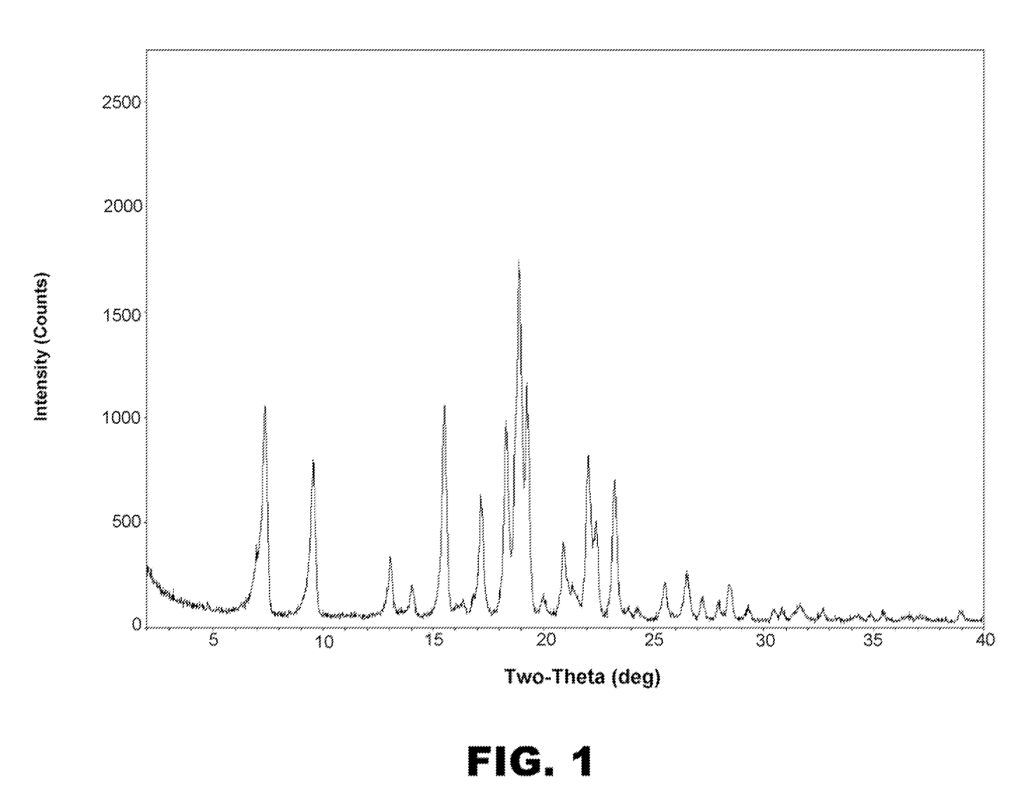

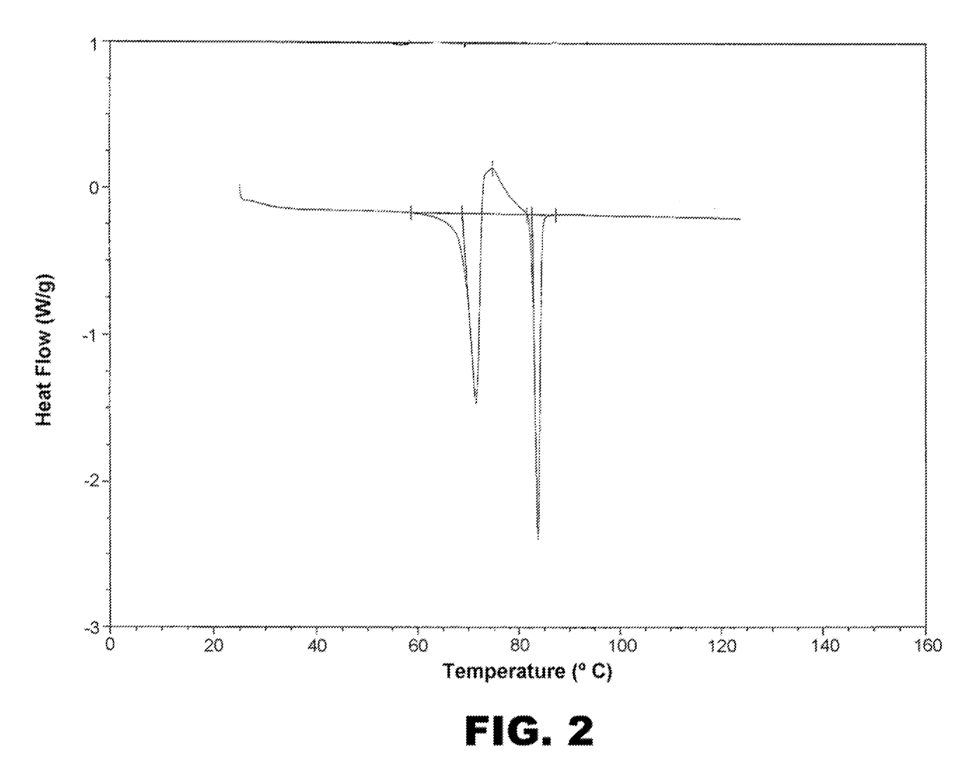

Crystalline fentanyl alkaloid may exist as Form I. Crystalline Form I of fentanyl alkaloid exhibits an X-ray powder diffraction pattern comprising characteristic peaks expressed in degrees 2-theta as diagrammed in FIG. 1. In particular, Form I exhibits predominant peaks expressed in degrees 2-theta at about 7.4, about 9.6, about 15.5, 18.9, and about 22.1. Form I also exhibits significant peaks at about 13.1, about 14.1, about 17.2, about 18.4, about 19.3, about 20.9, about 23.3, about 25.6, about 26.5, and about 28.5 degrees 2-theta. Crystalline Form I of fentanyl alkaloid exhibits a characteristic melting endoderm, as depicted in the differential scanning calorimetry thermogram shown in FIG. 2. In particular, crystalline Form I exhibits an endothermic transition with an onset of about 83°-85° C. as measured by differential scanning calorimetry (at a scan rate of 5° C. per minute).

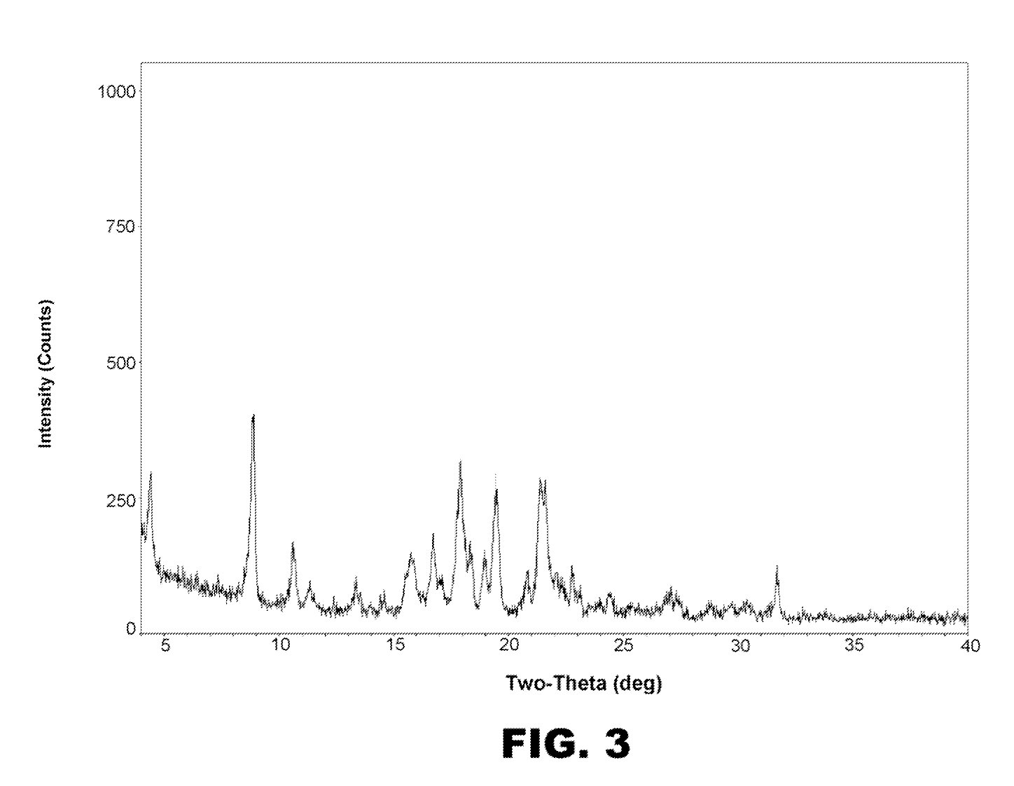

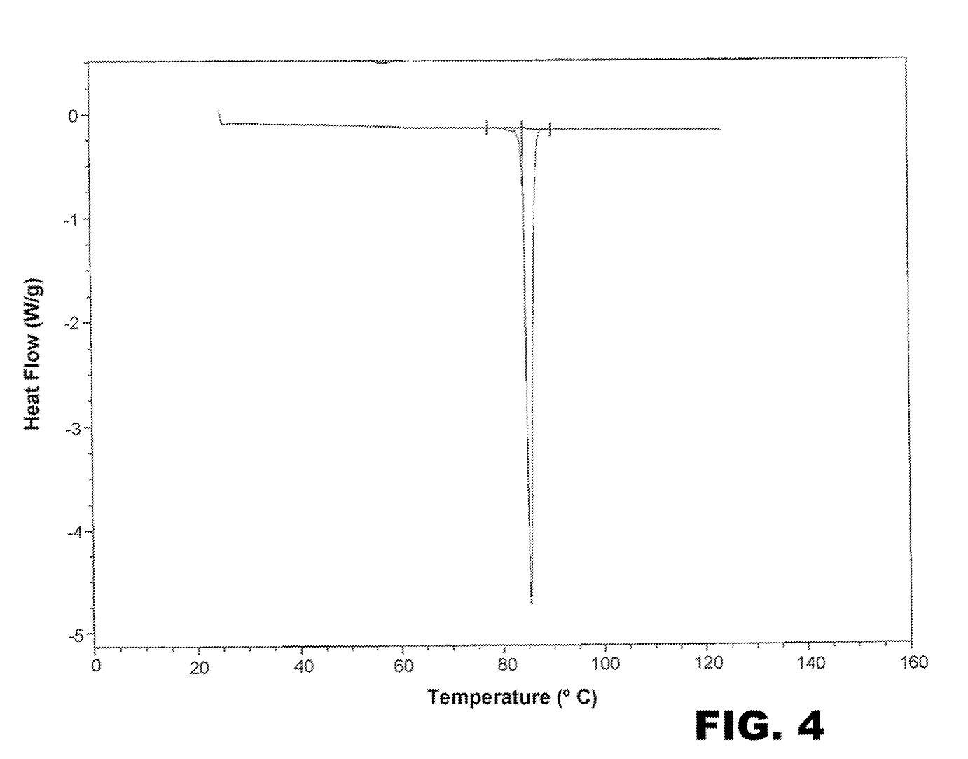

Fentanyl alkaloid crystals may also exist as crystalline Form II. This crystalline form exhibits an X-ray powder diffraction pattern comprising characteristic peaks expressed in degrees 2-theta as diagrammed in FIG. 3. In particular, Form II exhibits predominant peaks expressed in degrees 2-theta at about 8.9, about 17.9, about 19.4, and about 21.4. Form II also exhibits significant peaks at about 4.4, about 10.6, about 15.8, about 16.7, about 19.0, about 20.9, and about 31.7 degrees 2-theta. Crystalline Form II of fentanyl alkaloid exhibits a characteristic melting endoderm, as depicted in the differential scanning calorimetry thermogram shown in FIG. 4. In particular, crystalline Form I exhibits an endothermic transition with an onset of about 70°-73° C. as measured by differential scanning calorimetry (at a scan rate of 5° C. per minute).

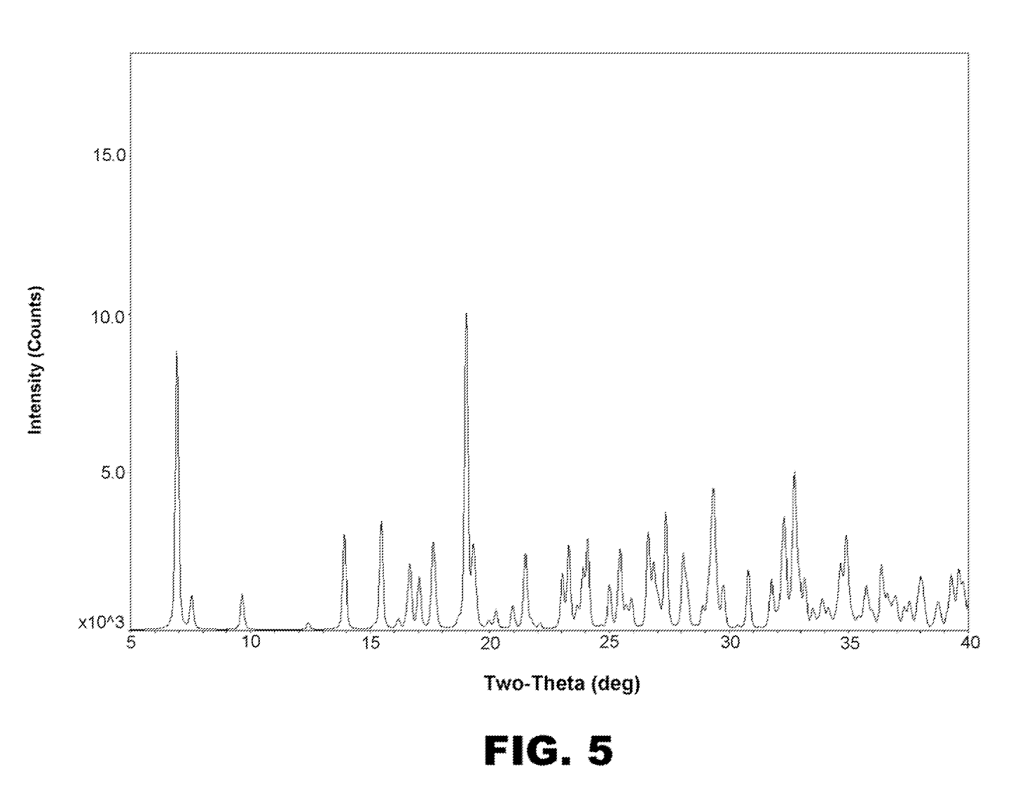

At extremely low temperatures, crystalline fentanyl alkaloid may exist as Form III. Crystalline Form III exhibits an X-ray powder diffraction pattern comprising characteristic peaks expressed in degrees 2-theta as diagrammed in FIG. 5. In particular, Form II exhibits predominant peaks expressed in degrees 2-theta at about 8.9, about 17.9, about 19.4, and about 21.4. Form III also exhibits significant peaks at about 14.0, about 15.5, about 17.7, about 23.3, about 24.1, about 26.5, about 27.4, about 32.3, and about 34.9.

In general, each of the crystalline forms of fentanyl alkaloid is substantially pure. The phrase “substantially pure,” as used herein, means that the crystalline form has a purity of about 95% by weight, or more preferably about 97% by weight, as defined by X-ray powder diffraction. Stated another way, the crystalline form has no more than about 5% by weight, or more preferably no more than about 3% by weight, of another form of fentanyl alkaloid.

### (II) Pharmaceutical Compositions

Another aspect of the invention provides for pharmaceutical compositions comprising crystalline Form II of fentanyl alkaloid and at least one pharmaceutically acceptable excipient. In general, the pharmaceutical composition will comprise an effective dosage amount of fentanyl alkaloid, i.e., an amount of fentanyl alkaloid sufficient to provide analgesia and/or anesthesia to the subject being administered the pharmaceutical composition. In some embodiments, the pharmaceutical composition may comprise substantially pure Form II of fentanyl alkaloid, as defined above. In other embodiments, the pharmaceutical composition may further comprise another crystalline or amorphous form of fentanyl alkaloid. For example, the pharmaceutical composition may further comprise crystalline Form I in addition to crystalline Form II of fentanyl alkaloid. The amount of Form II in such pharmaceutical compositions, therefore, may range from about 97%, about 95%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5%, or about 3% by weight of the total amount of fentanyl alkaloid.

A variety of excipients commonly used in pharmaceutical formulations may be selected on the basis of several criteria such as, e.g., the desired dosage form and the release profile properties of the dosage form. Non-limiting examples of suitable excipients include an agent selected from the group consisting of a binder, a filler, a non-effervescent disintegrant, an effervescent disintegrant, a preservative, a diluent, a flavoring agent, a sweetener, a lubricant, an oral dispersing agent, a coloring agent, a taste masking agent, a pH modifier, a stabilizer, a compaction agent, and combinations of any of these agents.

In one embodiment, the excipient may be a binder. Suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, polypeptides, peptides, and combinations thereof.

In another embodiment, the excipient may be a filler. Suitable fillers include carbohydrates, inorganic compounds, and polyvinilpirrolydone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, and sorbitol.

The excipient may be a non-effervescent disintegrant. Suitable examples of non-effervescent disintegrants include starches (such as corn starch, potato starch, and the like), pregelatinized and modified starches thereof, sweeteners, clays (such as bentonite), micro-crystalline cellulose, alginates, sodium starch glycolate, and gums (such as agar, guar, locust bean, karaya, pecitin, and tragacanth).

In another embodiment, the excipient may be an effervescent disintegrant. By way of non-limiting example, suitable effervescent disintegrants include sodium bicarbonate in combination with citric acid, and sodium bicarbonate in combination with tartaric acid.

The excipient may comprise a preservative. Suitable examples of preservatives include antioxidants (such as alpha-tocopherol or ascorbate) and antimicrobials (such as parabens, chlorobutanol or phenol). In other embodiments, an antioxidant such as butylated hydroxytoluene (BHT) or butylated hydroxyanisole (BHA) may be utilized.

In another embodiment, the excipient may include a diluent. Diluents suitable for use include pharmaceutically acceptable saccharides such as sucrose, dextrose, lactose, microcrystalline cellulose, fructose, xylitol, and sorbitol; polyhydric alcohols; starches; pre-manufactured direct compression diluents; and mixtures of any of the foregoing.

The excipient may include flavoring agents. Flavoring agents may be chosen from synthetic flavor oils and flavoring aromatics and/or natural oils, extracts from plants, leaves, flowers, fruits, and combinations thereof. By way of example, these may include cinnamon oils, oil of wintergreen, peppermint oils, clover oil, hay oil, anise oil, eucalyptus, vanilla, citrus oils (such as lemon oil, orange oil, grape and grapefruit oil), and fruit essences (such as apple, peach, pear, strawberry, raspberry, cherry, plum, pineapple, and apricot).

In another embodiment, the excipient may include a sweetener. By way of non-limiting example, the sweetener may be selected from glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; stevia-derived sweeteners; chloro derivatives of sucrose such as sucralose; sugar alcohols such as sorbitol, mannitol, sylitol, and the like. Also contemplated are hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof.

In another embodiment, the excipient may be a lubricant. Suitable non-limiting examples of lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

The excipient may be a dispersion enhancer. Suitable dispersants may include starch, alginic acid, polyvinylpyrrolidones, guar gum, kaolin, bentonite, purified wood cellulose, sodium starch glycolate, isoamorphous silicate, and microcrystalline cellulose.

Depending upon the embodiment, it may be desirable to provide a coloring agent. Suitable color additives include food, drug and cosmetic colors (FD&C), drug and cosmetic colors (D&C), or external drug and cosmetic colors (Ext. D&C). These colors or dyes, along with their corresponding lakes, and certain natural and derived colorants may be suitable for use in the present invention depending on the embodiment.

The excipient may include a taste-masking agent. Taste-masking materials include cellulose hydroxypropyl ethers (HPC); low-substituted hydroxypropyl ethers (L-HPC); cellulose hydroxypropyl methyl ethers (HPMC); methylcellulose polymers and mixtures thereof; polyvinyl alcohol (PVA); hydroxyethylcelluloses; carboxymethylcelluloses and salts thereof; polyvinyl alcohol and polyethylene glycol co-polymers; monoglycerides or triglycerides; polyethylene glycols; acrylic polymers; mixtures of acrylic polymers with cellulose ethers; cellulose acetate phthalate; and combinations thereof.

In various embodiments, the excipient may include a pH modifier. In certain embodiments, the pH modifier may include sodium carbonate or sodium bicarbonate.

The weight fraction of the excipient or combination of excipients in the pharmaceutical composition may be about 98% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the pharmaceutical composition.

The pharmaceutical compositions detailed herein may be manufactured in one or several dosage forms. Suitable dosage forms include transdermal systems or patches. The transdermal system may be a matrix system, a reservoir system, or a system without rate-controlling membranes. Other suitable dosage forms also include tablets, including suspension tablets, chewable tablets, effervescent tablets or caplets; pills; powders such as a sterile packaged powder, a dispensable powder, and an effervescent powder; capsules including both soft or hard gelatin capsules such as HPMC capsules; lozenges; a sachet; a sprinkle; a reconstitutable powder or shake; a troche; pellets such as sublingual or buccal pellets; granules; liquids for oral or parenteral administration; suspensions; emulsions; semisolids; or gels.

The dosage forms may be manufactured using conventional pharmacological techniques. Conventional pharmacological techniques include, e.g., one or a combination of methods: (1) dry mixing, (2) direct compression, (3) milling, (4) dry or non-aqueous granulation, (5) wet granulation, or (6) fusion. See, e.g., Lachman et al., The Theory and Practice of Industrial Pharmacy (1986). Other methods include, e.g., prilling, spray drying, pan coating, melt granulation, granulation, wurster coating, tangential coating, top spraying, extruding, coacervation and the like.

In general, the pharmaceutical compositions of the invention will be used for analgesia and anesthesia, most often in operating rooms, intensive care units, or palliative care units. The pharmaceutical compositions, and in particular transdermal delivery systems, may also be used for the management of oncologic and other chronic pain conditions.

The amount of active ingredient that is administered to a subject can and will vary depending upon a variety of factors such as the age and overall health of the subject, and the particular mode of administration. Those skilled in the art will appreciate that dosages may also be determined with guidance from Goodman & Goldman's The Pharmacological Basis of Therapeutics, Tenth Edition (2001), Appendix II, pp. 475-493, and the Physicians' Desk Reference.

### (III) Processes for Preparing Crystalline Forms of Fentanyl Alkaloid

Still another aspect of the invention provides processes for preparing substantially pure crystalline forms of fentanyl alkaloid. Crystalline Forms I and II may be obtained by crystallization, starting with a solution of fentanyl alkaloid, with each crystalline form resulting by crystallization from a different solvent. Processes are also provided for the conversion of crystalline Form I into crystalline Form II, and for the formation of an amorphous phase of fentanyl alkaloid.

(a) Processes for Preparing Crystalline Forms of Fentanyl Alkaloid

The process for preparing a substantially pure crystalline form of fentanyl alkaloid comprises (a) contacting fentanyl alkaloid with a solvent to form a saturated or near saturated solution, and (b) evaporating the solvent in the solution to form a mass of crystals of the substantially pure crystalline form of fentanyl alkaloid. The fentanyl alkaloid that is contacted with the solvent may be in a solid form (e.g., a powder) or a liquid form (e.g., in a solution comprising a co-solvent, or a concentrated oil/gel/gum).

The solvent used in the process can and will vary depending upon the embodiment. The solvent may be a protic solvent, an aprotic solvent, or a combination thereof. Suitable protic solvents include, but are not limited to, methanol, ethanol, isopropanol, n-propanol, isobutanol, n-butanol, s-butanol, t-butanol, water formic acid, acetic acid, or combinations thereof. Non-limiting examples of suitable aprotic solvents include acetone, acetonitrile, dichloromethane, tetrahydrofuran, or combinations thereof. In a preferred embodiment, the solvent may be a mixture of methanol and water, a mixture of ethanol and water, or a mixture of isopropanol and water. The weight ratio of alcohol to water may range from about 0.3:1 to about 3:1, or more preferably from about 0.7:1 to about 1.5:1. In preferred embodiments, the ratio of methanol to water may be about 1:1; the ratio of ethanol to water may be about 1:1; and the ratio of isopropanol to water may be about 1.5:1. The weight ratio of solvent to fentanyl alkaloid may range from about 5:1 to about 20:1, or more preferably from about 5:1 to about 10:1.

The process further comprises evaporating the solvent in the saturated or near saturated solution to form a mass of crystals of substantially pure crystalline fentanyl alkaloid. Typically, the evaporation is conducted slowly such that crystals are formed slowly. The rate of evaporation may be slowed by placing the saturated or near saturated solution in a flask with a narrow opening, covering the opening with paper or foil comprising a few small holes, or sealing the opening with a cap into which a needle has been inserted. Evaporation of the solvent may be conducted at atmosphere or in an inert environment (i.e., under nitrogen or argon). The solvent may be evaporated at atmospheric pressure or at a pressure that is less than atmospheric pressure.

The temperature of the process can and will vary. The temperature of step (a) may range from about 4° C. to about the boiling temperature of the solvent. In a preferred embodiment, step (a) of the process may be conducted at about room temperature. Step (b) of the process may be conducted at a temperature that ranges from about −10° C. to about 40° C., or more preferably from about 0° C. to about 25° C. In a preferred embodiment, step (b) of the process may be conducted at about room temperature.

The process generally further comprises collecting the crystals of the substantially pure crystalline form of fentanyl alkaloid. The crystals may be collected by filtration, centrifugation, or other techniques well known in the art. The process may further comprise drying the crystals of the substantially pure crystalline form of fentanyl alkaloid. The crystals may be dried under a vacuum either at room temperature or at an elevated temperature. The crystals may be identified or characterized using X-ray powder diffraction, differential scanning calorimetry, or another technique known to those of skill in the art.

In one preferred embodiment, the solvent is a mixture of methanol and water, the process is conducted at room temperature, and the crystalline Form I of fentanyl alkaloid is prepared.

In another preferred embodiment, the solvent is a mixture of ethanol and water, the process is conducted at room temperature, and crystalline Form l of fentanyl alkaloid is prepared.

In yet another preferred embodiment, the solvent is a mixture of isopropanol and water, the process is conducted at room temperature, and the crystalline Form II of fentanyl alkaloid is prepared.

(b) Process for Converting Form I Into Form II

The process for converting fentanyl alkaloid crystalline Form I into crystalline Form II comprises (a) melting crystalline Form I of fentanyl alkaloid, (b) cooling the melted fentanyl alkaloid from step (a), and (c) reheating the cooled fentanyl alkaloid from step (b) to form crystalline Form II.

As detailed above, crystalline Form I of fentanyl alkaloid exhibits a melting temperature of about 83-85° C. The conversion process comprises heating crystalline Form I to about 86°-90° C. The melted fentanyl alkaloid is then rapidly cooled to less than about −25° C. In a preferred embodiment, the melted fentanyl alkaloid may be cooled to about −50° C. The process further comprises reheating the cooled fentanyl alkaloid to above the glass transition phase, i.e., to about 30°-50° C., wherein the fentanyl alkaloid crystallizes as Form II. The resultant Form II crystals may be collected and characterized as described above.

(c) Process for Forming Amorphous Phase

The process for preparing an amorphous phase of fentanyl alkaloid comprises melting fentanyl alkaloid by heating it to about 86°-90° C. and then rapidly cooling the heated fentanyl alkaloid to less than about −25° C. The amorphous phase of fentanyl alkaloid may be characterized by differential scanning calorimetry.

### EXAMPLES

The following examples illustrate various embodiments of the invention.

### Example 1

### Characterization of Form I Crystals Formed by Slow Evaporation of Solvent 1

A saturated or near saturated solution of fentanyl alkaloid was prepared by mixing fentanyl alkaloid with a solution of a 1:1 ratio of ethanol and water (solvent 1). The solution was transferred to a small vial and sealed with a septa-cap. A needle was poked through the septa-cap and the vial was maintained at room temperature under nitrogen purge or at atmosphere. The needle allowed for slow evaporation and crystal growth. The crystals were collected and dried using standard procedures.

The crystals were characterized by X-ray powder diffraction spectrometry and differential scanning calorimetry (DCS). The diffraction pattern was obtained using a Bruker/Siemens D500 X-ray diffractometer, equipped with a graphite monochromator, and a Cu X-ray source operated at 40 kV, 30 mA, over the range of 2-40 degrees 2-theta. DCS was performed using a Q100 modulated differential scanning calorimeter (TA Instruments; New Castle, Del.) at a temperature range of 25-125° C. and scan rate of a 5° C. per minute (the instrument was calibrated using Indium).

The crystals were identified as having crystalline Form I. Form I is the predominant crystalline form in the fentanyl alkaloid produced by Mallinckrodt Inc. Table 1 summarizes the X-ray powder diffraction data for the Form I crystals, i.e., 2-theta degree positions of the peaks, height of the peaks, area of the peaks, and so forth. FIG. 1 presents the characteristic X-ray powder diffraction pattern for crystalline Form I. Crystalline Form I of fentanyl alkaloid exhibited predominant peaks at about 7.4, about 9.6, about 15.5, 18.9, and about 22.1 degrees 2-theta. FIG. 2 presents a DSC trace for crystalline Form I. Form I exhibited an endothermic transition with an onset of about 83°-85° C.

TABLE 1
X-Ray Powder Diffraction Spectral Lines of Form I.

| | | | Inten- | | Inten- | | |
|---|---|---|---|---|---|---|---|
| 2- | d Value | Back- | sity | | sity | | |
| Theta | (Å) | ground | Height | % | Area | % | FWHM* |
| 4.416 | 19.9928 | 137 | 160 | 46.7 | 1869 | 24.0 | .0198 |
| 8.897 | 9.9313 | 59 | 344 | 100.0 | 4816 | 61.8 | 0.238 |
| 10.619 | 8.3243 | 54 | 114 | 33.2 | 1314 | 16.9 | 0.196 |
| 11.340 | 7.7966 | 48 | 48 | 13.9 | 522 | 6.7 | 0.186 |
| 13.360 | 6.6222 | 39 | 66 | 19.3 | 906 | 11.6 | 0.232 |
| 15.759 | 5.6188 | 53 | 88 | 25.5 | 2030 | 26.0 | 0.393 |
| 16.719 | 5.2984 | 63 | 121 | 35.1 | 1662 | 21.3 | 0.234 |
| 17.076 | 5.1882 | 62 | 46 | 13.3 | 768 | 9.9 | 0.286 |
| 17.919 | 4.9461 | 58 | 261 | 76.0 | 5537 | 71.0 | 0.360 |
| 18.337 | 4.8342 | 55 | 114 | 33.3 | 2055 | 26.4 | 0.305 |
| 18.962 | 4.6764 | 50 | 103 | 30.0 | 1310 | 16.8 | 0.216 |
| 19.439 | 4.5627 | 47 | 245 | 71.1 | 3513 | 45.1 | 0.244 |
| 20.852 | 4.2566 | 56 | 60 | 17.5 | 622 | 8.0 | 0.176 |
| 21.381 | 4.1525 | 43 | 243 | 70.7 | 7795 | 100.0 | 0.546 |
| 22.080 | 4.0225 | 63 | 48 | 14.0 | 1359 | 17.4 | 0.480 |
| 22.734 | 3.9083 | 57 | 67 | 19.5 | 764 | 9.8 | 0.194 |
| 23.122 | 3.8436 | 45 | 43 | 12.5 | 393 | 5.0 | 0.155 |
| 23.967 | 3.7099 | 41 | 24 | 7.1 | 239 | 3.1 | 0.167 |
| 24.420 | 3.6421 | 39 | 36 | 10.5 | 610 | 7.8 | 0.287 |
| 26.858 | 3.3168 | 35 | 41 | 12.0 | 949 | 12.2 | 0.389 |
| 27.046 | 3.2942 | 34 | 49 | 14.3 | 1127 | 14.5 | 0.390 |
| 27.419 | 3.2502 | 35 | 33 | 9.6 | 528 | 6.8 | 0.271 |
| 28.723 | 3.1055 | 30 | 30 | 8.7 | 414 | 5.3 | 0.235 |
| 30.376 | 2.9402 | 36 | 26 | 7.7 | 321 | 4.1 | 0.206 |
| 31.678 | 2.8223 | 29 | 96 | 27.9 | 967 | 12.4 | 0.172 |

*FWHM = full width at half-maximum

### Example 2

### Characterization of Form I Crystals Formed by Slow Evaporation of Solvent 2

A saturated or near saturated solution of fentanyl alkaloid was prepared by mixing fentanyl alkaloid with a solution of a 1:1 ratio of methanol and water (solvent 2). The solution was transferred to a small vial and sealed with a septa-cap. A needle was poked through the septa-cap and the vial was maintained at room temperature under nitrogen purge or at atmosphere. The needle allowed for slow evaporation and crystal growth. The crystals were collected and dried using standard procedures. The crystals were characterized by X-ray powder diffraction spectrometry and differential scanning calorimetry essentially as detailed in Example 1. The crystals were identified as being crystalline Form I (see FIGS. 1 and 2, Table 1).

### Example 3

### Characterization of Form II Crystals Formed by Slow Evaporation of Solvent 3

A saturated or near saturated solution of fentanyl alkaloid was prepared by mixing fentanyl alkaloid with a solution of a 1.5:1 ratio of isopropanol and water (solvent 3). The solution was maintained at room temperature under nitrogen purge or at atmosphere. The needle allowed for slow evaporation and crystal growth. The crystals were collected and dried using standard procedures. The crystals were characterized by X-ray powder diffraction spectrometry and differential scanning calorimetry essentially as detailed in Example 1.

The crystals were identified as having crystalline Form II. Table 2 summarizes the X-ray powder diffraction data for the Form II crystals, i.e., 2-theta degree positions of the peaks, height of the peaks, area of the peaks, and so forth. FIG. 3 presents the characteristic X-ray powder diffraction pattern for crystalline Form II, which exhibited predominant peaks at about 8.9, about 17.9, about 19.4, and about 21.4 degrees 2-theta. FIG. 4 presents the DSC thermogram for crystalline Form II. Form II exhibited two endothermic transitions and an exothermic transition. The first endothermic transition, with an onset of about 70°-73° C., represents the melting temperature of Form II, the exothermic transition represents the crystallization temperature of Form I, and the second endothermic transition, with an onset of about 83°-85° C., represents the melting temperature of Form I.

TABLE 2
X-Ray Powder Diffraction Spectral Lines of Form II.

| | | | Inten- | | Inten- | | |
|---|---|---|---|---|---|---|---|
| 2- | d Value | Back- | sity | | sity | | |
| Theta | (Å) | ground | Height | % | Area | % | FWHM* |
| 4.416 | 19.9928 | 137 | 160 | 46.7 | 1869 | 24.0 | .0198 |
| 8.897 | 9.9313 | 59 | 344 | 100.0 | 4816 | 61.8 | 0.238 |
| 10.619 | 8.3243 | 54 | 114 | 33.2 | 1314 | 16.9 | 0.196 |
| 11.340 | 7.7966 | 48 | 48 | 13.9 | 522 | 6.7 | 0.186 |
| 13.360 | 6.6222 | 39 | 66 | 19.3 | 906 | 11.6 | 0.232 |
| 15.759 | 5.6188 | 53 | 88 | 25.5 | 2030 | 26.0 | 0.393 |
| 16.719 | 5.2984 | 63 | 121 | 35.1 | 1662 | 21.3 | 0.234 |
| 17.076 | 5.1882 | 62 | 46 | 13.3 | 768 | 9.9 | 0.286 |
| 17.919 | 4.9461 | 58 | 261 | 76.0 | 5537 | 71.0 | 0.360 |
| 18.337 | 4.8342 | 55 | 114 | 33.3 | 2055 | 26.4 | 0.305 |
| 18.962 | 4.6764 | 50 | 103 | 30.0 | 1310 | 16.8 | 0.216 |
| 19.439 | 4.5627 | 47 | 245 | 71.1 | 3513 | 45.1 | 0.244 |
| 20.852 | 4.2566 | 56 | 60 | 17.5 | 622 | 8.0 | 0.176 |
| 21.381 | 4.1525 | 43 | 243 | 70.7 | 7795 | 100.0 | 0.546 |
| 22.080 | 4.0225 | 63 | 48 | 14.0 | 1359 | 17.4 | 0.480 |
| 22.734 | 3.9083 | 57 | 67 | 19.5 | 764 | 9.8 | 0.194 |
| 23.122 | 3.8436 | 45 | 43 | 12.5 | 393 | 5.0 | 0.155 |
| 23.967 | 3.7099 | 41 | 24 | 7.1 | 239 | 3.1 | 0.167 |
| 24.420 | 3.6421 | 39 | 36 | 10.5 | 610 | 7.8 | 0.287 |
| 26.858 | 3.3168 | 35 | 41 | 12.0 | 949 | 12.2 | 0.389 |
| 27.046 | 3.2942 | 34 | 49 | 14.3 | 1127 | 14.5 | 0.390 |
| 27.419 | 3.2502 | 35 | 33 | 9.6 | 528 | 6.8 | 0.271 |
| 28.723 | 3.1055 | 30 | 30 | 8.7 | 414 | 5.3 | 0.235 |
| 30.376 | 2.9402 | 36 | 26 | 7.7 | 321 | 4.1 | 0.206 |
| 31.678 | 2.8223 | 29 | 96 | 27.9 | 967 | 12.4 | 0.172 |

*FWHM = full width at half-maximum

Example 4

### Conversion of Form I into Form II

Crystalline Form II of fentanyl alkaloid was also prepared by melting Form I crystals (i.e., heating to about 86°-90° C.). The melted fentanyl alkaloid was then rapidly cooled to about −50° C., and then reheated to about 30°-50° C. The crystals were collected using standard procedures. The crystals were characterized by X-ray powder diffraction spectrometry and differential scanning calorimetry essentially as described in Example 1. The newly formed crystals were of crystalline Form II (see FIGS. 3 and 4, Table 2).

### Example 4

### Conversion of Form I into Form III

A sample of Form I was rapidly cooled in liquid nitrogen (i.e., to about −196° C.). The resultant crystals were characterized by single crystal X-ray diffraction at liquid nitrogen temperatures using standard procedures, and then a powder X-ray pattern was calculated from the single crystal structure. The newly formed crystals were identified as having crystalline Form III. (Form III crystals were not observed at room temperature.)

Table 3 summarizes the X-ray powder diffraction data for the Form III crystals, i.e., 2-theta degree positions of the peaks, height of the peaks, area of the peaks, and so forth. FIG. 5 presents the characteristic X-ray powder diffraction pattern for crystalline Form III, which exhibited predominant peaks at about 7.0, about 19.1, about 29.4, and about 32.7 degrees 2-theta.

TABLE 3
X-Ray Powder Diffraction Spectral Lines of Form III.

| | | | Inten- | | Inten- | | |
|---|---|---|---|---|---|---|---|
| 2- | d Value | Back- | sity | | sity | | |
| Theta | (Å) | ground | Height | % | Area | % | FWHM* |
| 6.961 | 12.6892 | 31 | 8774 | 88.6 | 87092 | 84.2 | 0.169 |
| 7.579 | 11.6546 | 13 | 1061 | 10.7 | 12705 | 12.3 | 0.204 |
| 9.681 | 9.1283 | 9 | 1117 | 11.3 | 10878 | 10.5 | 0.166 |
| 12.440 | 7.1095 | 9 | 203 | 2.1 | 1816 | 1.8 | 0.152 |
| 13.958 | 6.3394 | 11 | 3012 | 30.4 | 30018 | 29.0 | 0.169 |
| 15.501 | 5.7120 | 32 | 3397 | 34.3 | 33655 | 32.5 | 0.168 |
| 16.218 | 5.4608 | 119 | 241 | 2.4 | 1692 | 1.6 | 0.119 |
| 16.699 | 5.3045 | 74 | 2008 | 20.3 | 22642 | 21.9 | 0.192 |
| 17.081 | 5.1869 | 98 | 1579 | 15.9 | 16031 | 15.5 | 0.173 |
| 17.679 | 5.0127 | 78 | 2688 | 27.1 | 29325 | 28.4 | 0.185 |
| 19.059 | 4.6529 | 98 | 9902 | 100.0 | 103430 | 100.0 | 0.178 |
| 19.340 | 4.5858 | 105 | 2625 | 26.5 | 63979 | 61.9 | 0.414 |
| 20.001 | 4.4357 | 106 | 208 | 2.1 | 2020 | 2.0 | 0.165 |
| 20.299 | 4.3713 | 89 | 540 | 5.4 | 4947 | 4.8 | 0.156 |
| 20.999 | 4.2272 | 75 | 678 | 6.8 | 6218 | 6.0 | 0.156 |
| 21.520 | 4.1259 | 55 | 2377 | 24.0 | 26969 | 26.1 | 0.193 |
| 22.120 | 4.0154 | 42 | 181 | 1.8 | 1701 | 1.6 | 0.160 |
| 23.061 | 3.8537 | 42 | 1757 | 17.7 | 19406 | 18.8 | 0.188 |
| 23.321 | 3.8112 | 42 | 2642 | 26.7 | 31167 | 30.1 | 0.201 |
| 24.119 | 3.6869 | 119 | 2768 | 28.0 | 37858 | 36.6 | 0.233 |
| 25.021 | 3.5559 | 118 | 1314 | 13.3 | 11997 | 11.6 | 0.155 |
| 25.461 | 3.4955 | 117 | 2463 | 24.9 | 28207 | 27.3 | 0.195 |
| 25.940 | 3.4321 | 116 | 893 | 9.0 | 14379 | 13.9 | 0.274 |
| 26.641 | 3.3434 | 136 | 2955 | 29.8 | 45159 | 43.7 | 0.260 |
| 26.880 | 3.3142 | 129 | 2022 | 20.4 | 57337 | 55.4 | 0.482 |
| 27.379 | 3.2548 | 147 | 3572 | 36.1 | 35505 | 34.3 | 0.169 |
| 28.101 | 3.1728 | 144 | 2282 | 23.0 | 30679 | 29.7 | 0.229 |
| 28.921 | 3.0848 | 143 | 637 | 6.4 | 16321 | 15.8 | 0.436 |
| 29.360 | 3.0396 | 109 | 4377 | 44.2 | 71837 | 69.5 | 0.279 |
| 29.779 | 2.9977 | 74 | 1326 | 13.4 | 18729 | 18.1 | 0.240 |
| 30.821 | 2.8988 | 79 | 1813 | 18.3 | 17713 | 17.1 | 0.166 |
| 31.782 | 2.8133 | 84 | 1510 | 15.2 | 16190 | 15.7 | 0.182 |
| 32.320 | 2.7677 | 84 | 3501 | 35.4 | 58583 | 56.6 | 0.284 |
| 32.741 | 2.7330 | 495 | 4534 | 45.8 | 63645 | 61.5 | 0.239 |
| 33.181 | 2.6978 | 361 | 1285 | 13.0 | 15654 | 15.1 | 0.207 |
| 33.523 | 2.6710 | 362 | 325 | 3.3 | 1989 | 1.9 | 0.104 |
| 33.921 | 2.6406 | 363 | 619 | 6.2 | 6955 | 6.7 | 0.191 |
| 34.180 | 2.6212 | 364 | 325 | 3.3 | 3487 | 3.4 | 0.182 |
| 34.700 | 2.5831 | 353 | 1771 | 17.9 | 36508 | 35.3 | 0.350 |
| 34.921 | 2.5672 | 349 | 2648 | 26.7 | 43543 | 42.1 | 0.280 |
| 35.760 | 2.5089 | 322 | 1076 | 10.9 | 14917 | 14.4 | 0.236 |
| 36.382 | 2.4674 | 308 | 1752 | 17.7 | 29614 | 28.6 | 0.287 |
| 36.980 | 2.4289 | 314 | 760 | 7.7 | 15646 | 15.1 | 0.350 |
| 37.560 | 2.3927 | 121 | 779 | 7.9 | 13414 | 13.0 | 0.293 |
| 38.040 | 2.3636 | 121 | 1561 | 15.8 | 24753 | 23.9 | 0.269 |
| 38.761 | 2.3213 | 121 | 770 | 7.8 | 9523 | 9.2 | 0.210 |
| 39.301 | 2.2906 | 121 | 1622 | 16.4 | 29886 | 28.9 | 0.313 |
| 39.620 | 2.2729 | 253 | 1700 | 17.2 | 39356 | 38.1 | 0.394 |
| 40.102 | 2.2467 | 412 | 1368 | 13.8 | 47692 | 46.1 | 0.593 |
| 40.641 | 2.2181 | 679 | 2295 | 23.2 | 25408 | 24.6 | 0.188 |
| 41.099 | 2.1945 | 256 | 1477 | 14.9 | 20419 | 19.7 | 0.235 |
| 41.521 | 2.1731 | 244 | 902 | 9.1 | 8585 | 8.3 | 0.162 |
| 42.002 | 2.1493 | 255 | 3010 | 30.4 | 32202 | 31.1 | 0.182 |
| 42.661 | 2.1177 | 237 | 2384 | 24.1 | 30263 | 29.3 | 0.216 |
| 43.520 | 2.0778 | 63 | 966 | 9.8 | 26633 | 25.7 | 0.469 |
| 44.322 | 2.0421 | 63 | 374 | 3.8 | 3738 | 3.6 | 0.170 |
| 44.740 | 2.0240 | 72 | 712 | 7.2 | 13813 | 13.4 | 0.330 |
| 45.940 | 1.9739 | 106 | 1299 | 13.1 | 20237 | 19.6 | 0.265 |
| 46.262 | 1.9609 | 126 | 1557 | 15.7 | 19243 | 18.6 | 0.210 |
| 47.202 | 1.9240 | 147 | 495 | 5.0 | 11285 | 10.9 | 0.387 |
| 47.842 | 1.8997 | 181 | 736 | 7.4 | 25165 | 24.3 | 0.582 |
| 48.121 | 1.8894 | 403 | 408 | 4.1 | 7934 | 7.7 | 0.331 |
| 48.821 | 1.8639 | 537 | 733 | 7.4 | 8233 | 8.0 | 0.191 |
| 49.360 | 1.8448 | 392 | 4423 | 44.7 | 64126 | 62.0 | 0.246 |
| 50.019 | 1.8220 | 748 | 4103 | 41.4 | 43136 | 41.7 | 0.179 |
| 50.720 | 1.7985 | 760 | 2280 | 23.0 | 38516 | 37.2 | 0.287 |
| 51.301 | 1.7794 | 225 | 1104 | 11.2 | 23814 | 23.0 | 0.367 |
| 51.619 | 1.7693 | 225 | 1178 | 11.9 | 22954 | 22.2 | 0.331 |
| 52.059 | 1.7553 | 225 | 541 | 5.5 | 5295 | 5.1 | 0.166 |
| 52.681 | 1.7361 | 241 | 1766 | 17.8 | 47526 | 45.9 | 0.457 |
| 53.039 | 1.7252 | 257 | 1197 | 12.1 | 40193 | 38.9 | 0.571 |
| 53.682 | 1.7060 | 268 | 1221 | 12.3 | 18229 | 17.6 | 0.254 |
| 54.036 | 1.6957 | 256 | 644 | 6.5 | 11337 | 11.0 | 0.299 |
| 54.781 | 1.6744 | 242 | 1178 | 11.9 | 21302 | 20.6 | 0.307 |
| 55.322 | 1.6593 | 335 | 1727 | 17.4 | 48524 | 46.9 | 0.478 |
| 55.722 | 1.6483 | 548 | 453 | 4.6 | 4462 | 4.3 | 0.167 |
| 56.939 | 1.6159 | 504 | 755 | 7.6 | 20032 | 19.4 | 0.451 |
| 57.221 | 1.6086 | 522 | 790 | 8.0 | 13510 | 13.1 | 0.291 |
| 57.719 | 1.5959 | 540 | 1212 | 12.2 | 18509 | 17.9 | 0.260 |
| 58.357 | 1.5800 | 531 | 641 | 6.5 | 6376 | 6.2 | 0.169 |
| 58.781 | 1.5696 | 530 | 1518 | 15.3 | 19668 | 19.0 | 0.220 |
| 59.422 | 1.5542 | 512 | 334 | 3.4 | 5941 | 5.7 | 0.303 |
| 59.920 | 1.5425 | 512 | 1902 | 19.2 | 25952 | 25.1 | 0.232 |
| 60.601 | 1.5267 | 419 | 1758 | 17.8 | 23560 | 22.8 | 0.228 |
| 61.539 | 1.5057 | 323 | 583 | 5.9 | 10246 | 9.9 | 0.299 |
| 62.279 | 1.4896 | 360 | 997 | 10.1 | 11220 | 10.8 | 0.191 |
| 63.318 | 1.4676 | 358 | 345 | 3.5 | 2936 | 2.8 | 0.145 |
| 63.714 | 1.4595 | 372 | 381 | 3.8 | 6388 | 6.2 | 0.285 |
| 64.679 | 1.4400 | 387 | 770 | 7.8 | 8748 | 8.5 | 0.193 |
| 65.417 | 1.4255 | 489 | 613 | 6.2 | 4584 | 4.4 | 0.127 |
| 65.600 | 1.4220 | 399 | 449 | 4.5 | 9695 | 9.4 | 0.367 |
| 66.202 | 1.4105 | 401 | 732 | 7.4 | 8907 | 8.6 | 0.207 |
| 67.320 | 1.3898 | 318 | 1287 | 13.0 | 18601 | 18.0 | 0.246 |
| 67.940 | 1.3786 | 309 | 549 | 5.5 | 6551 | 6.3 | 0.203 |
| 68.280 | 1.3725 | 364 | 724 | 7.3 | 14201 | 13.7 | 0.334 |
| 69.101 | 1.3582 | 485 | 1147 | 11.6 | 10487 | 10.1 | 0.155 |
| 69.560 | 1.3504 | 434 | 984 | 9.9 | 13631 | 13.2 | 0.235 |
| 70.179 | 1.3400 | 484 | 485 | 4.9 | 7397 | 7.2 | 0.259 |
| 70.901 | 1.3281 | 571 | 1193 | 12.1 | 14799 | 14.3 | 0.211 |
| 71.682 | 1.3155 | 615 | 454 | 4.6 | 7447 | 7.2 | 0.279 |
| 72.221 | 1.3070 | 706 | 712 | 7.2 | 10385 | 10.0 | 0.248 |
| 72.922 | 1.2962 | 568 | 393 | 4.0 | 2450 | 2.4 | 0.106 |
| 73.781 | 1.2832 | 529 | 910 | 9.2 | 16616 | 16.1 | 0.310 |
| 74.520 | 1.2723 | 578 | 1083 | 10.9 | 18632 | 18.0 | 0.292 |
| 75.282 | 1.2613 | 568 | 715 | 7.2 | 14522 | 14.0 | 0.345 |
| 75.741 | 1.2548 | 317 | 733 | 7.4 | 15565 | 15.0 | 0.361 |
| 76.201 | 1.2484 | 518 | 836 | 8.4 | 6785 | 6.6 | 0.138 |
| 77.561 | 1.2298 | 317 | 766 | 7.7 | 28173 | 27.2 | 0.625 |
| 77.900 | 1.2253 | 661 | 707 | 7.1 | 16234 | 15.7 | 0.390 |
| 78.599 | 1.2162 | 319 | 1126 | 11.4 | 16577 | 16.0 | 0.250 |
| 78.840 | 1.2131 | 319 | 1830 | 18.5 | 23621 | 22.8 | 0.219 |
| 79.520 | 1.2044 | 395 | 1218 | 12.3 | 15075 | 14.6 | 0.210 |
| 79.798 | 1.2009 | 484 | 835 | 8.4 | 12803 | 12.4 | 0.261 |
| 80.262 | 1.1951 | 319 | 1166 | 11.8 | 12359 | 11.9 | 0.180 |
| 80.559 | 1.1914 | 812 | 1763 | 17.8 | 15392 | 14.9 | 0.148 |
| 81.300 | 1.1825 | 822 | 998 | 10.1 | 14353 | 13.9 | 0.245 |
| 82.101 | 1.1729 | 693 | 1406 | 14.2 | 14699 | 14.2 | 0.178 |
| 82.380 | 1.1697 | 620 | 1145 | 11.6 | 16162 | 15.6 | 0.240 |
| 83.741 | 1.1541 | 508 | 1043 | 10.5 | 16195 | 15.7 | 0.264 |
| 84.400 | 1.1468 | 531 | 1264 | 12.8 | 13506 | 13.1 | 0.182 |
| 84.979 | 1.1404 | 306 | 1063 | 10.7 | 21306 | 20.6 | 0.341 |
| 85.561 | 1.1341 | 220 | 878 | 8.9 | 9629 | 9.3 | 0.186 |
| 85.841 | 1.1312 | 220 | 588 | 5.9 | 6134 | 5.9 | 0.177 |
| 86.860 | 1.1205 | 220 | 869 | 8.8 | 26391 | 25.5 | 0.516 |
| 87.321 | 1.1158 | 220 | 1161 | 11.7 | 35725 | 34.5 | 0.523 |
| 87.938 | 1.1095 | 642 | 436 | 4.4 | 5455 | 5.3 | 0.213 |
| 88.500 | 1.1039 | 666 | 1088 | 11.0 | 12008 | 11.6 | 0.188 |
| 88.779 | 1.1012 | 676 | 781 | 7.9 | 10699 | 10.3 | 0.233 |
| 89.419 | 1.0949 | 790 | 520 | 5.2 | 2547 | 2.5 | 0.083 |

*FWHM = full width at half-maximum

### Example 5

### Formation of an Amorphous Form of Fentanyl Alkaloid

A sample of fentanyl alkaloid was melted and then cooled to less than about −25° C. The amorphous phase was characterized by DSC, essentially as described in Example 1 except that the lower temperature range was reduced to about −20° C. It was found that the amorphous form exhibited a sub-ambient glass transition at about −15° C.

What is claimed is:

1. A crystalline form of fentanyl alkaloid, N-[1-(2-phenylethyl)-4-piperidyl]-N-phenylpropanamide, the crystalline form being Form II.

2. The crystalline form of claim 1, wherein Form II exhibits an X-ray powder diffraction pattern having characteristic peaks expressed in degrees 2-theta at about 8.9, about 17.9, about 19.4, and about 21.4.

3. The crystalline form of claim 1, wherein Form II exhibits an endothermic transition with an onset of about 70°-73° C. as measured by differential scanning calorimetry.

4. The crystalline form of claim 1, wherein Form II comprises no more than about 5% by weight of another form of fentanyl alkaloid.

5. A pharmaceutical composition, the composition comprising crystalline Form II of fentanyl alkaloid, N-[1-(2-phenylethyl)-4-piperidyl]-N-phenylpropanamide, and at least one pharmaceutically acceptable excipient.

6. The pharmaceutical composition of claim 5, wherein crystalline Form II exhibits an X-ray powder diffraction pattern having characteristic peaks expressed in degrees 2-theta at about 8.9, about 17.9, about 19.4, and about 21.4, and an endothermic transition with an onset of about 70°-73° C. as measured by differential scanning calorimetry.

7. The pharmaceutical composition of claim 5, further comprising another crystalline or amorphous form of fentanyl alkaloid.

8. The pharmaceutical composition of claim 5, further comprising crystalline Form I of fentanyl alkaloid.

9. The pharmaceutical composition of claim 8, wherein Form I exhibits an X-ray powder diffraction pattern having characteristic peaks expressed in degrees 2-theta at about 7.4, about 9.6, about 15.5, 18.9 and about 22.1, and an endothermic transition with an onset of about 83°-85° C. as measured by differential scanning calorimetry.

10. A process for preparing a substantially pure crystalline form of fentanyl alkaloid, N-[1-(2-phenylethyl)-4-piperidyl]-N-phenylpropanamide, the process comprising:
a) contacting fentanyl alkaloid with a solvent to form a saturated or near saturated solution; and
b) evaporating the solvent in the solution to form a mass of crystals of the substantially pure crystalline form.

11. The process of claim 10, wherein the solvent is slowly evaporated in an inert atmosphere.

12. The process of claim 10, further comprising the step of collecting the crystals of the substantially pure crystalline form.

13. The process of claim 12, further comprising the step of drying the crystals of the substantially pure crystalline form.

14. The process of claim 10, wherein the solvent is selected from the group consisting of a protic solvent selected from the group consisting of methanol, ethanol, isopropanol, n-propanol, isobutanol, n-butanol, t-butanol, water, formic acid, and acetic acid; an aprotic solvent selected from the group consisting of acetone, acetonitrile, dichloromethane, and tetrahydrofuran; and combinations thereof.

15. The process of claim 10, wherein the solvent is a mixture of methanol and water, and the crystalline form is Form I, which exhibits an X-ray powder diffraction pattern having characteristic peaks expressed in degrees 2-theta at about 7.4, about 9.6, about 15.5, 18.9, and about 22.1, and an endothermic transition with an onset of about 83°-85° C. as measured by differential scanning calorimetry.

16. The process of claim 10, wherein the solvent is a mixture of ethanol and water, and the crystalline form is Form I, which exhibits an X-ray powder diffraction pattern having characteristic peaks expressed in degrees 2-theta at about 8.9, about 17.9, about 19.4, and about 21.4, and an endothermic transition with an onset of about 83°-85° C. as measured by differential scanning calorimetry.

17. The process of claim 10, wherein the solvent is a mixture of isopropanol and water, and the crystalline form is Form II, which exhibits an X-ray powder diffraction pattern having characteristic peaks expressed in degrees 2-theta at 8.9, about 17.9, about 19.4, and about 21.4, and an endothermic transition with an onset of about 70°-73° C. as measured by differential scanning calorimetry.

18. A process for converting a crystalline Form I of fentanyl alkaloid into a crystalline Form II of fentanyl alkaloid, the process comprising:
a) melting the crystalline Form I of fentanyl alkaloid;
b) cooling the fentanyl alkaloid from step a); and
c) heating the fentanyl alkaloid from step b) to form the crystalline Form II of fentanyl alkaloid.

19. The process of claim 18, wherein step a) is conducted at a temperature of about 86° C. to about 90° C.

20. The process of claim 19, wherein step b) is conducted at a temperature of less than about −25° C.

21. The process of claim 20, wherein step c) is conducted at a temperature of about 30° C. to about 50° C.

22. The process of claim 18, wherein the crystalline Form I exhibits an X-ray powder diffraction pattern having characteristic peaks expressed in degrees 2-theta at about 7.4, about 9.6, about 15.5, 18.9, and about 22.1, and an endothermic transition with an onset of about 83°-85° C. as measured by differential scanning calorimetry.

23. The process of claim 18, wherein the crystalline Form II exhibits an X-ray powder diffraction pattern having characteristic peaks expressed in degrees 2-theta at about 8.9, about 17.9, about 19.4, and about 21.4, and an endothermic transition with an onset of about 70°-73° C. as measured by differential scanning calorimetry.
